(19) United States
(12) Patent Application Publication
(10) Pub. No.: US 2003/0176908 A1
(43) Pub. Date: Sep. 18, 2003

(54) Water-impregnable electrode for electrical stimulation device

(76) Inventor: Hsin-Yi Lin, Hsin-Tien (TW)

Correspondence Address: Hsin-Yi Lin, P. O. Box 55-846, Taipei, 104, TW

(21) Appl. No.: 10/104,776

(22) Filed: Mar. 18, 2002

(51) Int. Cl. A61N 1/04

(52) U.S. Cl. 607/142

(57) ABSTRACT

A water-impregnable electrode includes: a contacting layer made of water-penetrable electrically conductive materials to be contacted with a user's (or patient's) skin, a water-impregnated intermediate layer made of water-absorbable materials contiguous to the contacting layer for soaking up water or moisture into the intermediate layer, a backing layer made of electrically insulative materials contiguous to the intermediate layer for covering the intermediate layer as sandwiched in between the contacting layer and the backing layer, and an electrical conducting device leading through the layers as combined together for electrically connecting the contacting layer to an electrical stimulator for directing the electrical pulses from the stimulator into the user's body (skin) for stimulating the user's nerve or muscle for medical, health, exercise or toning purposes.

### BACKGROUND OF THE INVENTION:

A conventional transcutaneous nerve or muscle stimulation electrode may be provided for stimulating a patient's body while impulse signals are electrically conducted into the body. An electrically conductive adhesive is applied on a fabric or medium of the electrode for adhering the electrode to the patient's skin. However, once the adhesive is cured or dried, the electrode will be disposed and can not be repeatedly used to thereby cause waste of electrode material and also cause environmental problem. The conductive adhesive or gel as applied on the user's skin is so sticky to easily contaminate the user's body or clothes, thereby causing dislike by the user.

The present inventor has found the drawbacks of the conventional electrode and invented the present electrode for stimulation device.

### SUMMARY OF THE INVENTION

The object of the present invention is to provide a water-impregnable electrode including: a contacting layer made of water-penetrable electrically conductive materials to be contacted with a user's (or patient's) skin, a water-impregnated intermediate layer made of water-absorbable materials contiguous to the contacting layer for soaking up water or moisture into the intermediate layer, a backing layer made of electrically insulative materials contiguous to the intermediate layer for covering the intermediate layer as sandwiched in between the contacting layer and the backing layer, and an electrical conducting device leading through the layers as combined together for electrically connecting the contacting layer to an electrical stimulator for directing the electrical pulses from the stimulator into the user's body (skin) for stimulating the user's nerve or muscle for medical, health, exercise or toning purposes.

### BRIEF DESCRIPTION OF THE DRAWING

FIG. 1 is a sectional drawing of an electrode of the present invention.

FIG. 2 is a bottom view of the electrode of FIG. 1.

FIG. 3 is a sectional drawing of another preferred embodiment of the present invention.

FIG. 4 is a partially cut-away bottom view of the electrode as shown in FIG. 3.

FIG. 5 is an illustration of the electrode with the carrier and fastening members of the present invention.

FIG. 6 shows still another preferred embodiment of the present invention.

FIG. 7 shows further preferred embodiment of the present invention for double electrodes formed on a carrier member.

FIG. 8 shows still further preferred embodiment of the present invention for quadruple electrodes on a carrier member.

### DETAILED DESCRIPTION

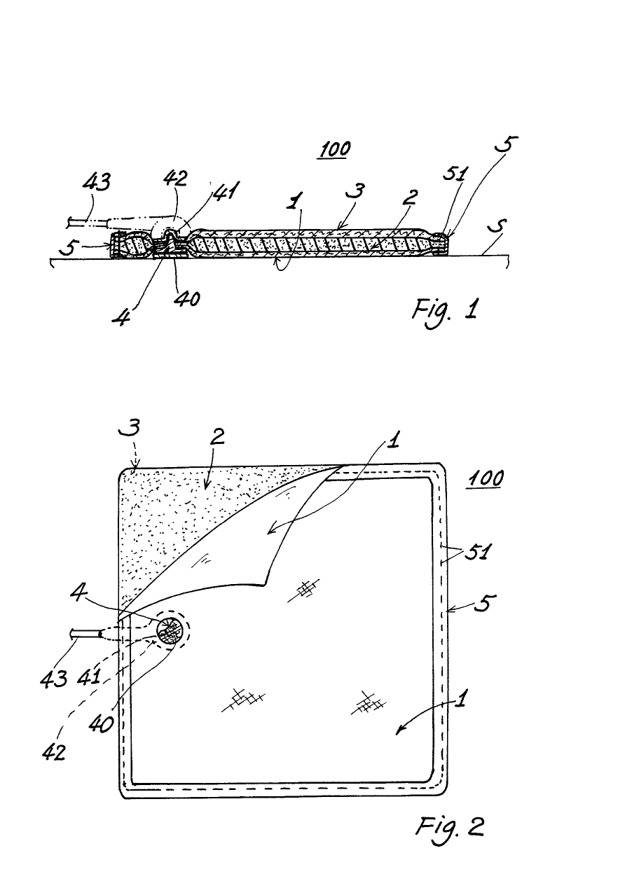

As shown in FIGS. 1 and 2, the electrode or electrode unit 100 of the present invention comprises: a contacting layer 1; a water-impregnated intermediate layer 2, a backing layer 3; an electrical conducting means 4 leading through the layers 1, 2, 3; and a rim member 5 circumferentially disposed around the edge portion of the layers 1, 2, 3 for combining or fastening all the layers 1, 2, 3 together for forming the electrode or electrode unit 100. All layers 1, 2, 3 should be made of soft flexible materials.

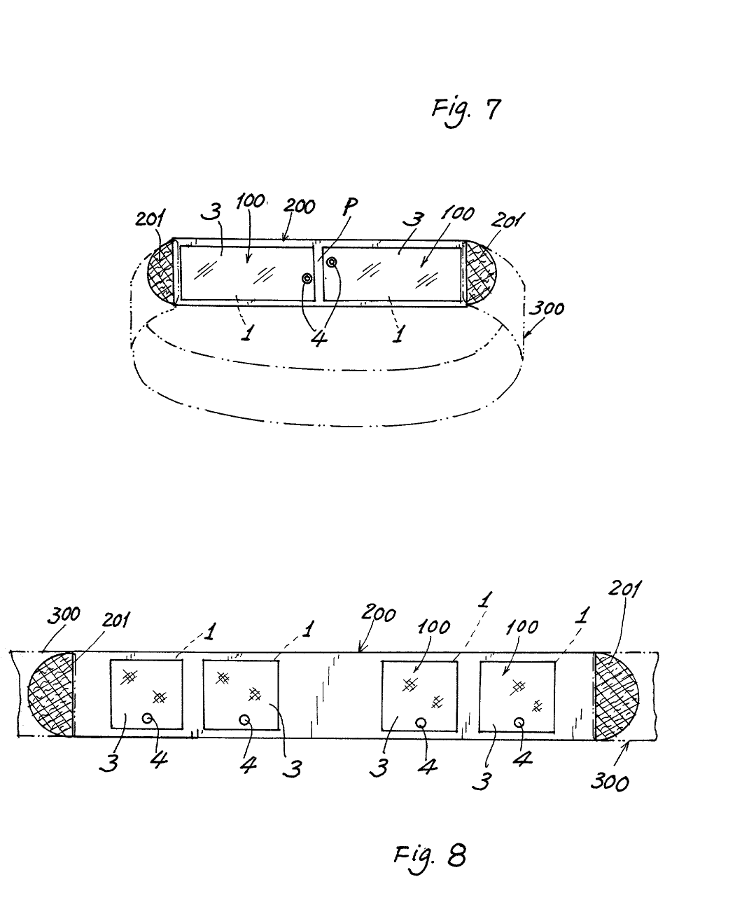

The electrodes 100 can be made individually as shown in FIGS. 1˜6; or a plurality of electrodes 100 may be juxtapositionally formed on a carrier member 200 as shown in FIGS. 7 and 8.

The electrode or electrodes 100 may be electrically connected to an electrical stimulator or a toning device for medical, health, exercise and toning purposes. The electrical connection methods are so conventional and are not described in detail in this invention. The number and shapes of electrodes as connected to a stimulator are not limited in the present invention.

The contacting layer 1 is made of water-penetrable porous or net-like materials having porosity and capillaries formed therein for penetrating water therethrough and also made of electrically conductive materials. It may be formed as: a carbon-fiber fabric, a textile fabric, a plastic film, a rubber film, a metallic layer and a printing sheet, all being electrically conductive.

The water-impregnated intermediate layer 2 is made of water-absorbable materials, such as: sponge, foam, cotton or fibers having porosity and capillaries existing therein for soaking up water or absorbing moisture into the intermediate layer 2 in an amount durable for a time period so as to penetrate water into the contacting layer 1 through capillaries existing in the intermediate layers 2 and in the contacting layer 1 to moisten the contacting layer 1 when performing the stimulation by an electrical stimulator (not shown).

The water-impregnated intermediate layer 2 may also serve as a “cushioning pad” as packed on a back or rear portion of the contacting layer 1 to render a comfortable cushioning when contacting and stimulating a user's (or patient's) skin or body portion and to also provide a uniform “touching” of the contacting layer 1 of the electrode 100 of this invention on the user's skin, which in turn provides a uniform electric current or charges distribution on the user's skin area to increase the stimulation or toning efficiency of the present invention.

The backing layer 3 adjacent to the intermediate layer 2 forms a protective cover of the intermediate layer and the contacting layer 1 of the electrode 100 of the present invention; and the backing layer 3 is made of electrically insulative materials such as plastic, rubber, insulative textile fabric, foam, elastomers, leather, and so on, not limited in the present invention.

The backing layer 3 may be formed as single layer or multiple layers composed of a plurality of layers of same or different electrically insulative materials, which may be formed as composite, or lamination combined or secured together to form the backing layer 3, not limited in this invention.

The electrical conducting means 4 includes: an electrically-conductive buckle device as shown in FIGS. 1, 2 inserted through the backing layer 3, the intermediate layer 2 and the contacting layer 1 for fastening the backing layer 3, the intermediate layer 2 and the contacting layer 1 together; and includes: a plug (or button) 41 protruding outwardly (or upwardly) from the backing layer 3 adapted to be connected with a snap female connector (or button socket) 42 having a wire 43 connectable to an electrical stimulator or toning device (not shown) for directing electrical pulses from the stimulator or toning device through the electrically-conductive buckle device 4 into the user's skin S (FIG. 1) for stimulation or toning operation.

An electrically insulative cover 40 is provided to insulatively cover a bottom portion of the electrically-conductive buckle device 4 to prevent a “point-stimulation” of the buckle device 4 and to allow the current from the buckle device 4 to be uniformly distributed into the whole contacting layer 1 especially when moistened by water as penetrated from the water content as impregnated in the intermediate layer 2. Otherwise, the current will largely flow into the patient's skin through the “single point” at the bottom of the buckle device 4 if the insulative cover 40 is not provided. The cover 40 may have a same color as that of the contacting layer 1, but not limited.

The rim member 5 is circumferentially disposed around the edge portion or contour of the electrode 100 to “fence” the edge portion of the electrode 100, with the rim member 5 having a cross section of U shape for encasing each edge portion of the backing layer 3, the intermediate layer 2 and the contacting layer 1 and a stitch 51 is sewn in the rim member 5 for firmly fastening and joining the backing layer 3, the intermediate layer 2 and the contacting layer 1.

Naturally, other fastening or joining methods may be applied in this invention (such as by embedding a flexible ring on the edge portion of the layers) by those having skill in the art.

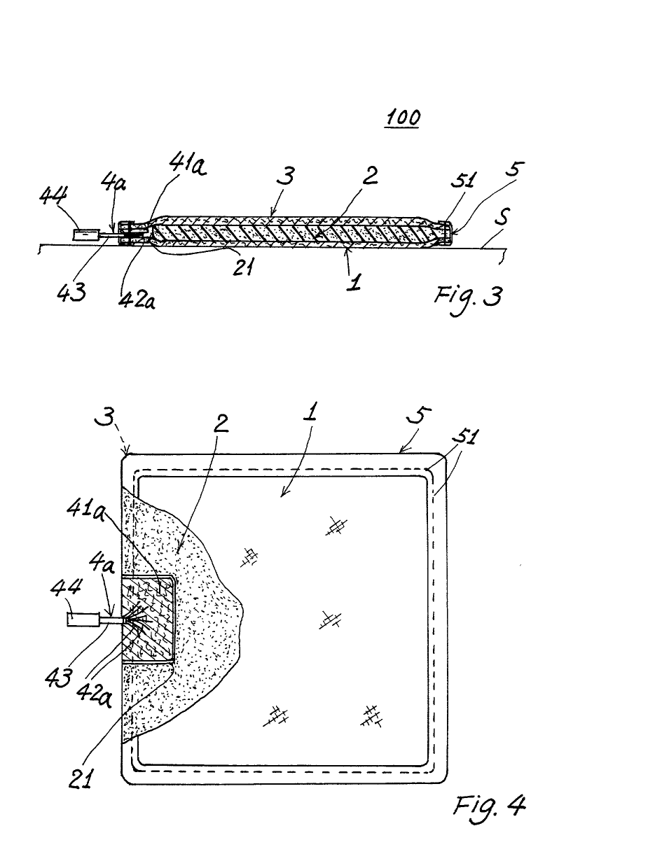

As shown in FIGS. 3 and 4, the present invention is modified to provide an electrical conducting means 4a to substitute the aforementioned buckle device 4 for the situations wherever the stitching operation may be obstructed or interfered by the metallic buckle device 4.

The electrical conducting means 4a of FIG. 3 and 4 includes: an electrically conductive chip (or pad or fabric) 41a adhered or secured on a bottom portion of the backing layer 3, a contactor portion 42a formed on an end of a wire 43 and electrically contacted with the electrically conductive chip 41a by sawing threads through the contacting layer 1, the chip 41a and the backing layer 3 through a notch (or window) 21 as cut out in the intermediate layer 2, and a connector 44 formed on the wire 43 to be connectable to a stimulator or toning device; whereby the electric current is led through the wire 43, the contactor portion 42a and the contacting layer 1 to stimulate the user's skin or body portion. (Note: The notch or window 21 is cut out for a well fastening and current connection of the conducting means 4a with the contacting layer 1).

The contactor portion 42a is sandwiched in between the chip 41a and the contacting layer 1 as firmly fastened by the stitch 51 as sewn in the rim member 5, thereby enhancing a tightly fastening for the electrical connecting elements (42a, 41a, 1) for a reliable current conducting through the conducting means 4a.

The conducting means 4a may also be modified to be an electrical connector, including snap male and female connectors, as electrically connected between the contacting layer 1 and the stimulator.

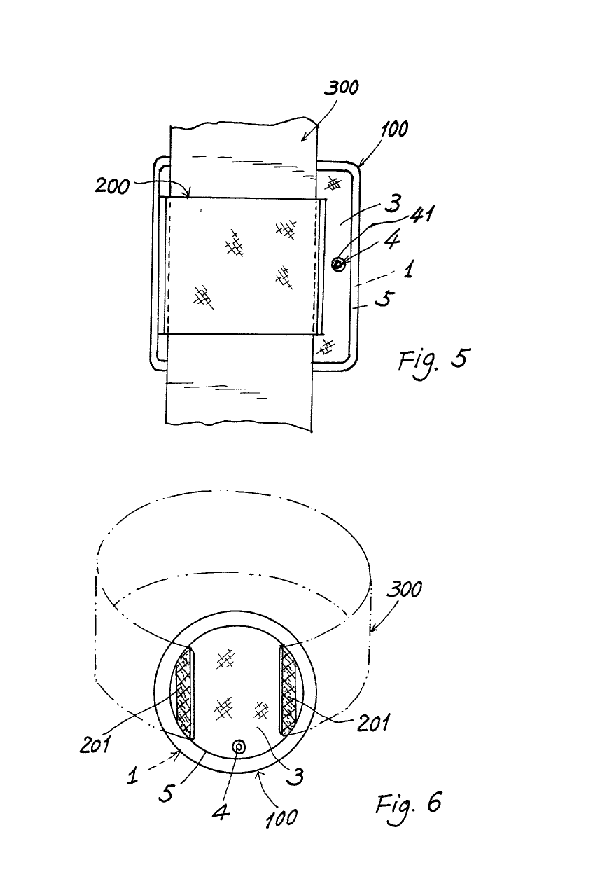

An example as shown in FIG. 5, a carrier member 200 such as a fastening loop is formed on the backing layer 3 for passing a fastening member 300 through the carrier member (the loop) 200 so that the electrode 100 of the present invention may be firmly fastened to a user's (patient's) body portion such as arm, leg, or other body portions (not shown).

As shown in FIG. 6, a round-shape electrode 100 is formed in accordance with the present invention, having Velcro tapes (or fasteners) 201 formed on the backing layer 3 of the electrode 100 adapted to be coupled to a fastening member 300 having Velcro tape (or fastener) also formed on the fastening member 300.

As shown in FIG. 7, two electrode units 100 are parallelly formed on a carrier member 200 which may be a belt or a strip having Velcro tapes 201 formed on opposite ends of the carrier member 200, whereby upon coupling with a fastening ember 300, the electrodes 100 on the carrier member 200 may then be fastened to a user's body portion for stimulation or toning operation.

As shown in FIG. 8, four electrodes 100 are parallelly or juxtapositionally formed on the carrier member 200 which is then coupled to a fastening member 300 to be fastened to a user's body.

The electrode 100 as formed on the carrier member 200 as shown in FIGS. 7, 8 may be made by extending the backing layer 3 of the electrode 100 to be the carrier member 200, with each electrode 100 being electrically insulative to any neighboring electrode 100 (such as numeral “P” as marked on FIG. 7).

Accordingly, each electrode or electrode unit 100 may be integrally formed with the carrier member 200, or each electrode 100 is fixed on the carrier member 200 by any conventional joining methods, not limited in the present invention.

Although FIGS. 7 and 8 illustrate the electrode number to be two and four electrodes respectively. However, the number of electrodes and their electrical connection (connected between the patient and the stimulators) methods are not limited in this invention.

The present invention is superior to a conventional electrode with the following advantages:

1. The electrodes can be repeatedly used for saving cost and for better environmental protection. Once the moisture is dried, water can be “re-filled” or soaked into the electrode to be impregnated by the intermediate layer 2. So, the intermediate layer 2 will play a role like a “water reservoir” to reserve water to continuously moisten the contacting layer 1 during stimulation or toning operation.

2. The water is penetrated into the porosity of the contacting layer 1 for uniformly distributing the electric current or charges to the whole area of the contacting layer 1 for a uniform comfortable stimulation since water (which is not pure or deionized water) is electrically conductive.

3. The intermediate layer 2, besides its water-reserving function, will serve as a cushioning pad as backed on the contacting layer 1 for elastically cushioning the contacting layer 1 for comfortably cushioning the user's (patient's) body when performing the stimulation. The “cushioning pad” of the intermediate layer 2 may be enhanced especially when a fastening member 300 such as a fastening belt resiliently fastens the backing layer 3 to force the intermediate layer 2 to thereby tightly cushion the contacting layer 1 for a uniform pressure distribution, uniform current (charge) distribution and also uniform water distribution, greatly increasing the stimulation (or toning) efficiency.

The present invention may be modified without departing from the spirit and scope of the present invention.

I claim:

1. An electrode for stimulation device comprising:
a contacting layer to be contacted with a user's body and made of water-penetrable porous materials having porosity and capillaries formed therein for penetrating water therethrough and made of electrically conductive materials;
a water-impregnated intermediate layer made of water-absorbable materials, having porosity and capillaries formed therein for soaking up water into the intermediate layer to penetrate water into the contacting layer 1 to moisten the contacting layer when performing a stimulation by an electrical stimulator; said water-impregnated intermediate layer packed on a back portion of the contacting layer to serve as cushioning pad when contacting and stimulating a user's body portion;
a backing layer adjacent to the intermediate layer covering the intermediate layer and the contacting layer and made of electrically insulative materials; and
an electrical conducting means inserted through the backing layer, the intermediate layer and the contacting layer for fastening the backing layer, the intermediate layer and the contacting layer together; and connected to an electrical stimulator or a toning device for directing electrical pulses from the stimulator or toning device through the electrically-conductive means into the user's body for stimulation or toning operation.

2. An electrode according to claim 1, wherein said electrical conducting means is an electrically conductive buckle device, including a connector formed on said conducting means to be connected with a stimulator.

3. An electrode according to claim 2, wherein said electrically conductive buckle device includes an electrically insulative cover insulatively covering a bottom portion of the electrically-conductive buckle device to allow a current led through the buckle device to be uniformly distributed into the contacting layer.

4. An electrode according to claim 1, wherein said electrode includes a rim member circumferentially disposed around an edge portion of all said layers to fence the edge portion, with the rim member encasing an edge portion of each said backing layer, said intermediate layer and said contacting layer, and a stitch sewn in the rim member for firmly fastening and joining the backing layer, the intermediate layer and the contacting layer.

5. An electrode according to claim 1, wherein said electrical conducting means includes: an electrically conductive chip secured on a bottom portion of the backing layer, a contactor portion formed on an end of a wire and electrically contacted with the electrically conductive chip by sawing threads through the contacting layer, the chip and the backing layer through a notch as cut out in the intermediate layer, and a connector formed on the wire to be connectable to a stimulator or toning device; whereby the electric current is led through the wire, the contactor portion and the contacting layer to stimulate the user's body.

6. An electrode according to claim 1, wherein said backing layer is formed on a carrier member for passing a fastening member through the carrier member to be firmly fastened to a user's body.

7. An electrode according to claim 1, wherein said back layer has a Velcro tape formed on the backing layer adapted to be coupled to a fastening member.

8. An electrode according to claim 1, wherein a plurality of said electrodes are parallelly formed on a carrier member coupled to a fastening member adapted to be fastened to a user's body.

9. An electrode according to claim 1, wherein said contacting layer is made of electrically conductive carbon-fiber fabric, electrically conductive textile fabric, electrically conductive plastics and rubbers, and electrically conductive metallic layer, all having porosity formed therein.

10. An electrode according to claim 1, wherein said intermediate layer is made of foam, sponge, cotton, and fibers having porosity formed therein and being water absorbable.

11. An electrode according to claim 1, wherein said backing layer is made of electrically insulative plastic, rubber, elastomer, foam and fabric materials.

12. An electrode according to claim 1, wherein said electrode is formed on a carrier member, said carrier member being made by extending the backing layer of the electrode to be integrally formed with the carrier member.
